(19) United States
(12) Patent Application Publication
(10) Pub. No.: US 2024/0316134 A1
(43) Pub. Date: Sep. 26, 2024

(54) BLOOD FLOW INCREASING AGENT FOR DORSOLATERAL PREFRONTAL CORTEX, DORSOLATERAL PREFRONTAL CORTEX ACTIVATOR, FOOD FOR INCREASING BLOOD FLOW IN DORSOLATERAL PREFRONTAL CORTEX, AND FOOD FOR ACTIVATING DORSOLATERAL PREFRONTAL CORTEX

(71) Applicant: KINJIRUSHI CO., LTD., Nagoya-shi, Aichi (JP)

(72) Inventors: Ryuta Kawashima, Sendai-shi (JP); Rui Nouchi, Sendai-shi (JP); Isao Okunishi, Nagoya-shi (JP)

(21) Appl. No.: 18/573,049

(22) PCT Filed: Mar. 29, 2023
(86) PCT No.: PCT/JP2023/012912

§ 371 Date: Dec. 21, 2023

(30) Foreign Application Priority Data
Mar. 30, 2022 (JP) 2022-057070

(51) Int. Cl. A61K 36/31; A61K 9/00; A61K 9/48; A61K 31/26; A61P 25/28

(52) U.S. Cl. CPC A61K 36/31; A61K 9/0053; A61K 9/4866; A61K 31/26; A61P 25/28

(57) ABSTRACT

A blood flow increasing agent for dorsolateral prefrontal cortex includes 6-methylsulfinylhexyl isothiocyanate.

### CROSS-REFERENCE TO RELATED APPLICATION

This international application claims the benefit of Japanese Patent Application No. 2022-057070 filed on Mar. 30, 2022 with the Japan Patent Office, and the entire disclosure of Japanese Patent Application No. 2022-057070 is incorporated in this international application by reference.

### TECHNICAL FIELD

The present disclosure relates to a blood flow increasing agent for dorsolateral prefrontal cortex, a dorsolateral prefrontal cortex activator, a food for increasing blood flow in the dorsolateral prefrontal cortex, and a food for activating the dorsolateral prefrontal cortex.

### BACKGROUND ART

Enhancement, maintenance, or improvement of cognitive function is sought by a wide range of generations, from the elderly to the young. Enhancement and the like of cognitive function is known to be related to increased activity of each brain part (for example, increased cerebral blood flow).

Dorsolateral prefrontal cortex is an area in an upper part of a prefrontal area of brain. The dorsolateral prefrontal cortex is known to be involved in working memory, which is a type of cognitive function (Non-Patent Document 1).

In recent years, compositions for increasing brain activity have been developed. For example, it is shown in Patent Document 1 that a composition consisting primarily of specific flavanol activates brain areas, such as right dorsolateral prefrontal cortex, in tasks for evaluating cognitive function.

### PRIOR ART DOCUMENTS

### Patent Document

Patent Document 1: Unexamined Japanese Patent Application Publication No. 2009-539991

### Non-Patent Document

Non-patent Document 1: Olesen, P., Westerberg, H. & Klingberg, T. Increased prefrontal and parietal activity after training of working memory. Nat Neurosci 7, 75-79 (2004)

### SUMMARY OF THE INVENTION

### Problems to be Solved by the Invention

The inventors have made every effort to seek for a new substance that can increase blood flow in dorsolateral prefrontal cortex.

It is desirable that one aspect of the present disclosure provides a novel blood flow increasing agent for the dorsolateral prefrontal cortex.

### Means for Solving the Problems

One aspect of the present disclosure provides a blood flow increasing agent for increasing blood flow in dorsolateral prefrontal cortex. The blood flow increasing agent comprises 6-methylsulfinylhexyl isothiocyanate.

In one aspect of the present disclosure, the blood flow increasing agent for dorsolateral prefrontal cortex may improve and/or enhance cognitive function by increasing the blood flow in the dorsolateral prefrontal cortex.

In one aspect of the present disclosure, the cognitive function may be episodic memory.

In one aspect of the present disclosure, the cognitive function may be working memory.

In one aspect of the present disclosure, the blood flow increasing agent for dorsolateral prefrontal cortex may be administered as a single dose.

In one aspect of the present disclosure, the blood flow increasing agent for dorsolateral prefrontal cortex may be orally administered.

In one aspect of the present disclosure, the 6-methylsulfinylhexyl isothiocyanate may be extracted from wasabi or synthesized.

One aspect of the present disclosure provides a blood flow increasing agent for dorsolateral prefrontal cortex comprising a wasabi extract.

One aspect of the present disclosure provides a food for increasing blood flow in dorsolateral prefrontal cortex. The food contains a blood flow increasing agent for dorsolateral prefrontal cortex.

One aspect of the present disclosure provides an activator for activating dorsolateral prefrontal cortex. The activator comprises 6-methylsulfinylhexyl isothiocyanate.

One aspect of the present disclosure provides a food for activating dorsolateral prefrontal cortex. The food contains a dorsolateral prefrontal cortex activator.

### Effects of the Invention

The composition as above provides a blood flow increasing agent for dorsolateral prefrontal cortex. In addition, the blood flow increasing agent improves and/or enhances cognitive function (for example, episodic memory or working memory) by increasing blood flow in the dorsolateral prefrontal cortex.

### BRIEF DESCRIPTION OF THE DRAWINGS

FIG. 1 shows graphs illustrating score evaluation results of Logical Memory (immediate), Logical Memory (delayed), and Face-and-Name Associative Memory. In each graph, the vertical axis of the graph shows the amount of change in scores of before and after lifestyle intervention. The amount of change is expressed by mean value±standard error. In each graph, the graph's bar on the left shows a test formulation group, and the graph's bar on the right shows a control formulation group.

FIG. 2 is a graph illustrating score evaluation results of Digit Span (Backward Span). The vertical axis of the graph shows the amount of change in scores of before and after the lifestyle intervention. The amount of change is expressed by mean value±standard error. The graph's bar on the left shows the test formulation group, and the graph's bar on the right shows the control formulation group.

FIG. 3 is a schematic diagram illustrating a method of an N-back task.

FIG. 4 is a graph illustrating calculation results of Rata Correct Scores in a 2-back task of before intake, 30 minutes after intake, and 60 minutes after intake of a formulation. The vertical axis of the graph shows the Rata Correct Scores. Each Score is expressed by a mean value. At each time point, the graph's bar on the left shows the test formulation group, and the graph's bar on the right shows the control formulation group.

FIG. 5 is a graph illustrating measurement results of brain activity in left dorsolateral prefrontal cortex during the 2-back task of before intake, 30 minutes after intake, and 60 minutes after intake of the formulation. The vertical axis of the graph shows measured values of brain activity. Each measured value is expressed by a mean value. At each time point, the graph's bar on the left shows the test formulation group, and the graph's bar on the right shows results of the control formulation group.

### MODE FOR CARRYING OUT THE INVENTION

A blood flow increasing agent of the present disclosure comprises isothiocyanates. In the present disclosure, isothiocyanates refer to a compound having a —NCS group. Isothiocyanates have, for example, an aromatic or aliphatic group as a side chain. An example of isothiocyanates having an aromatic group includes phenethyl isothiocyanate. Examples of isothiocyanates having an aliphatic group include one or more selected from a group consisting of isopropyl isothiocyanate, isobutyl isothiocyanate, 2-butyl isothiocyanate, isoamyl isothiocyanate, amyl isothiocyanate, allyl isothiocyanate, 3-butenyl isothiocyanate, 4-pentenyl isothiocyanate, 5-hexenyl isothiocyanate, 6-heptenyl isothiocyanate, 3-methylthiopropyl isothiocyanate, 4-methylthiobutyl isothiocyanate, S-methylthiopentyl isothiocyanate, 6-methylthiohexyl isothiocyanate, 7-methylthioheptyl isothiocyanate, 4-methylsulfinylbutyl isothiocyanate, 5-methylsulfinyl pentyl isothiocyanate, 6-methylsulfinylhexyl isothiocyanate, and 7-methylsulfinylheptyl isothiocyanate. Preferably, the blood flow increasing agent of the present disclosure comprises 4-methylsulfinylbutyl isothiocyanate, 5-methylsulfinylpentyl isothiocyanate, 6-methylsulfinylhexyl isothiocyanate, or 7-methylsulfinylheptyl isothiocyanate. Most preferably, the blood flow increasing agent of the present disclosure comprises 6-methylsulfinylhexyl isothiocyanate. Hereinafter, the blood flow increasing agent comprising 6-methylsulfinylhexyl isothiocyanate according to the present disclosure will be discussed in detail.

The blood flow increasing agent of the present disclosure comprises 6-methylsulfinylhexyl isothiocyanate (6-Methylsulfinyhexyl isothiocyanate) (abbreviated as 6-MSITC).

The blood flow increasing agent of the present disclosure increases blood flow in dorsolateral prefrontal cortex (DLPFC). In the present disclosure, “blood flow increase” refers to an increase in the amount of blood entering a unit volume of tissue per unit time. In other words, a blood flow increase in the DLPFC refers to an increase in the amount of blood entering a unit volume of the DLPFC per unit time. The blood flow increase in the DLPFC can be correlated with an increase in DLPFC activity.

The blood flow increasing agent of the present disclosure improves and/or enhances cognitive function by increasing blood flow in the DLPFC. In the present disclosure, “improve” means to make a favorable change in function or ability, or to inhibit or delay a decline in function or ability. In the present disclosure, “enhance” means to potentiate or advance function or ability. In the present disclosure, “cognitive function” means an advanced function of brain to correctly grasp the states of surroundings and oneself in order to act appropriately based on external information obtained through vision, hearing, and the like. The cognitive function in the present disclosure may be any cognitive function in which the DLPFC is involved, and examples thereof include episodic memory, working memory, executive function, attention, visuospatial ability, and processing ability. “Episodic memory” herein refers to memories of events experienced by an individual with accompanying information such as time, place, and emotion, and an ability to recall the events experienced by the individual with the accompanying information. “Working memory” refers to an ability to execute a series of tasks while temporarily keeping information. “Executive function” refers to an ability to maintain appropriate actions and thoughts for achieving future goals. “Attention” refers to an ability to direct attention to specific matters, or ability to maintain the attention. “Visuospatial ability” refers to an ability to process information that is visually taken in and recognize the whole image of space. “Processing ability” refers to an ability to process new information.

Preferably, the blood flow increasing agent of the present disclosure may be used to improve and/or enhance episodic memory and/or working memory by increasing blood flow in the DLPFC.

Various reports have been made regarding a relationship between the episodic and working memories and brain parts involved in the memories, and brain parts that are activated for the respective memories have been identified. For example, it is known that the DLPFC is responsible for working memory (Olesen, P et.al., Nat Neurosci., 7, 75-79, 2004). It is also known that frontal lobe (for example, the DLPFC and fronto-orbital area) and hippocampus are responsible for episodic memory (M. Natasha Rajah et.al., Brain., Volume 128, Issue 9, Pages 1964-1983 September 2005, and Georg Gron et.al., Neurobiology of Aging, Volume 24, Issue 3, Pages 453-462, 2003, etc.). Thus, episodic memory and/or working memory can be improved and/or enhanced by increasing blood flow in the DLPFC (enhancing DLPFC activity).

6-MSITC used in the blood flow increasing agent of the present disclosure can be extracted and purified from plants. Examples of the plants may include plants such as Bataceae (Bataccae), Brassicaceae (Brassicaceae), Bretschneideraceae (Bretschneideraceae), Capparaceae (Capparaceae), Caricaceae (Caricaceae), Euphorbiaceac (Euphorbiaceae), Gyrostemonaceae (Gyrostemonaceae), Limnanthaceae (Limnanthaceae), Moringaceae (Moringaceae), Pentadiplandraceae (Pentadiplandraceae), Phytolaccaceae (Phytolaccaceae), Pittosporaceae (Pittosporaceae), Resedaceae (Resedaceae), Salvadoraceae (Salvadoraceae), Tovariaceae (Tovariaceae), and Tropacolaceae (Tropaeolaceae). Specifically, examples of the plants include wasabi (Wasabia japonica) [also known as hon-wasabi], horseradish (Armoracia rusticana) [also known as yama-wasabi], Batis maritima (Japanese name unknown), Japanese mustard (Brassica juncea), broccoli (Brassica oleracea var. italica), mouse-ear cress (Arabidopsis thaliana), shepherd's purse (Capsella bursa-pastoris), watercress (Nasturtium officinale), Bretschneidera sinensis (Japanese name unknown), caper (Capparis spinosa), papaya (Carica papaya), Drypetes roxburghii (Japanese name unknown); Putranjiva roxburghii (Japanese name unknown), Tersonia brevipes (Japanese name unknown), Limnanthes douglasii (Japanese name unknown), horseradish tree (Moringa oleifera), Pentadiplandra brazzeana (Japanese name unknown), pokeweed (Phytolacca americana), Bursaris spinosa var. incana (Japanese name unknown), white mignonette (Reseda alba), Salvadora persica (Japanese name unknown), Tovaria pendula (Japanese name unknown), and garden nasturtium (Tropaeolum majus). However, 6-MSITC that can be used in the present disclosure is not limited to 6-MSITC obtained from the aforementioned plants, and all natural resources containing 6-MSITC can be used as raw materials.

It is preferable that 6-MSITC is extracted from wasabi.

6-MSITC used in the blood flow increasing agent of the present disclosure can be obtained by a chemical synthesis method. Details of the chemical synthesis method of 6-MSITC are described as follows.

The method by Kjaer et al. is followed in principle (Kjaer et al., Acta chem., Scand., 11, 1298, 1957) which discloses a common method for synthesizing isothiocyanate. Specifically, 6-chlorohexanol is used and refluxed with CH₃—SNa to obtain 6-methylthiohexanol. 6-methylthiohexanol is reacted with thionyl chloride (SOCl₂) to obtain 6-chlorohexyl methyl sulfide. Next, using the known Gabriel method as a method for synthesizing primary amines, an amino group is introduced by phthalimide potassium salt to produce N-(6-methylthio hexyl)-phthalimide. Hydrazine hydrate is added and refluxed with N-(6-methylthio hexyl)-phthalimide so as to obtain 6-methylthio hexyl amine. Then, thiocarbonyl chloride is reacted with 6-methylthio hexyl amine to obtain 6-methylthio hexyl isothiocyanate.

Further, a methylthio group in the obtained 6-methylthio hexyl isothiocyanate is oxidized with m-chloroperbenzoic acid to obtain 6-methylsulfinylhexyl isothiocyanate (6-MSITC) (Morimitsu et.al., J. Biol. Chem., 277, 3456, 2002).

The blood flow increasing agent of the present disclosure may contain a wasabi extract. Preferably, the wasabi extract contains 6-MSITC.

The blood flow increasing agent of the present disclosure may be contained in foods, cosmetics, pharmaceuticals, or quasi-drugs.

The form of foods containing the blood flow increasing agent of the present disclosure may be in any form, and is not limited. Specific examples of food forms include general foods, general drinks, supplements, health foods, foods with function claims, foods with health claims and special purpose foods such as foods for specified health use, soft drinks, tea drinks, health drinks, alcoholic beverages such as wines, confectioneries, cooked rices, breads, noodles, side dishes, and seasonings.

The form of cosmetics, pharmaceuticals, and quasi-drug containing the blood flow increasing agent of the present disclosure may be in any form, and is not limited. Specific examples of cosmetics, pharmaceuticals, and quasi-drugs include internal/external preparations, skin lotions, emulsions, creams, ointments, packs, skin cleansers, shampoos, hair conditioners, and bath additives.

It is reported in a past research that, in cultured cells, 6-MSITC increases VEGF that facilitates angiogenesis (Food Science and Technology Research, 24(3), 567-572, 2018). It is considered that application of 6-MSITC to scalp, for example, can facilitate peripheral angiogenesis, and accordingly blood flow in brain (for example, the DLPFC) can be increased.

The dosage form of cosmetics, pharmaceuticals, and quasi-drugs containing the blood flow increasing agent of the present disclosure is not limited. Specific examples of dosage forms of cosmetics, pharmaceuticals, and quasi-drugs include capsules, tablets, powders, granules, solutions, emulsions, creams, gels, ointments, sheets, and mousses.

In addition to 6-MSITC, the principal component, the blood flow increasing agent of the present disclosure may contain, as necessary, various optional components as secondary components to a suitable extent that the effect of the present disclosure is not impaired. The optional components are those generally used in foods, cosmetics, pharmaceuticals, and quasi-drugs.

The blood flow increasing agent of the present disclosure can be administered in various administration manners, but oral administration is preferred.

Dosage of the blood flow increasing agent of the present disclosure is determined in consideration of, for example, the age, sex, weight, usage, and dose of a subject. Daily dosage of 6-MSITC in the blood flow increasing agent, when orally administered, is preferably 0.01 mg/day to 10 mg/day, more preferably 0.05 mg/day to 5.0 mg/day, and desirably 0.1 mg/day to 1 mg/day. The dosage of the blood flow increasing agent in these numerical ranges can increase blood flow in the DLPFC of a subject.

When the wasabi extract is used as the blood flow increasing agent, the wasabi extract may be taken such that the daily dose of 6-MSTIC in the wasabi extract is 0.01 mg/day to 10 mg/day. It is preferable that the wasabi extract is taken such that the daily dose of 6-MSTIC in the wasabi extract is 0.01 mg/day to 10 mg/day, and desirably 0.1 mg/day to 1 mg/day.

The blood flow increasing agent of the present disclosure may be administered as a single dose. The blood flow increasing agent of the present disclosure can increase blood flow in the DLPFC of a subject by being administered only once to the subject.

The blood flow increasing agent of the present disclosure may be administered repeatedly on a long term or a short term basis. The frequency of dose is not limited and may be, for example, three times a day, twice a day, once a day, once every two days, once every three days, once a week, once every two weeks, and once a month. The period of administration is not limited, and may be one day, two days, three days, one week, two weeks, one month, half a year, one year, or longer.

The blood flow increasing agent of the present disclosure can be administered to subjects of various ages. The subjects may be elderly individuals (for example, 60 years old and over) and/or young individuals (for example, 10 to 30 years old).

In addition, one aspect of the present disclosure relates to a DLPFC activator for activating the DLPFC comprising 6-MSTIC. Activation of the DLPFC may include, in addition to a blood flow increase in the DLPFC as described above, various responses related to enhancement of DLPFC activity. Examples of responses related to enhancement of DLPFC activity include changes in electrical activity (specifically, brain waves), changes in magnetic fields, and changes in neurotransmitters that occur with neural activity in brain.

In addition, one aspect of the present disclosure relates to a method for increasing blood flow in the DLPFC in which the method includes administrating a blood flow increasing agent to a subject. The method for increasing blood flow may improve and/or enhance cognitive function by increasing blood flow in the DLPFC.

The blood flow increasing agent comprising 6-MSITC and/or the DLPFC activator for activating the DLPFC comprising 6-MSITC according to the present disclosure have been described above in detail. However, other isothiocyanates can be also used for the blood flow increasing agent and/or the DLPFC activator of the present disclosure. For example, the blood flow increasing agent of the present disclosure may comprise 4-methylsulfinylbutyl isothiocyanate. The DLPFC activator for activating the DLPFC according to the present disclosure may comprise 4-methylsulfinylbutyl isothiocyanate. The blood flow increasing agent comprising 4-methylsulfinylbutyl isothiocyanate and/or the DLPFC activator for activating the DLPFC comprising 4-methylsulfinylbutyl isothiocyanate can exhibit an effect comparable to the effect of the blood flow increasing agent comprising 6-MSITC and/or the effect of the DLPFC activator for activating the DLPFC comprising 6-MSITC.

### Examples

### Example 1: Preparation of Formulations

To study the effect of administration to humans on cognitive function, the formulations shown in Table 1 below were prepared as supplements to be taken by target subjects. The difference between the test formulation and the control formulation is whether Wasabi-sulfinyl (registered trademark) which is a component extracted from hon-wasabi is contained.

TABLE 1
Prepared Test Formulation and Control Formulation

| Type | Test formulation | Control formulation |
|---|---|---|
| Shape | Hard capsule | Hard capsule |
| Raw materials | Hon-wasabi extract, | Cyclodextrin, gelatin, |
| | cyclodextrin, gelatin, | caramel coloring |
| | caramel coloring | |
| Wasabi-sulfinyl | 100 mg/capsule | 0 mg/capsule |
| (registered | (Wasabi-sulfinyl | |
| trademark) content | (registered trademark) | |
| | contains 0.8% of | |
| | 6-MSITC) | |

| | |
|---|---|
| Preservation Method | Refridgerated |
| Provider of studied | KINJIRUSHI Co., Ltd. |
| food | |

TABLE 2
Nutrients of Test Formulation
and Control Formulation

| | Test | | Control | |
|---|---|---|---|---|
| Nutrient (per 100 g) | formulation | | formulation | |
| Water | 7.1 | g | 8.2 | g |
| Protein | 20.0 | g | 17.1 | g |
| Fat | 6.1 | g | 5.3 | g |
| Ash | 2.7 | g | 2.4 | g |
| Carbohydrate | 64.1 | g | 67.0 | g |
| Energy | 391 | kcal | 384 | kcal |
| Sodium | 44.1 | mg | 34.5 | mg |
| Sodium chloride | 0.112 | g | 0.08765 | g |
| equivalent | | | | |

### Example 2: Evaluation of Cognitive Function in Elderly Individuals

Using the test formulation and the control formulation prepared in Example 1, a long-term intervention test was conducted in the elderly individuals. Details are given below.

### (1) Flow of Test

The subjects were assigned to a test formulation group and a control formulation group using stratified randomization in consideration of the sex of the subjects. The test in Example 2 was conducted in the order of a preliminary explanation (explanation about cognitive function assessments and a questionnaire-based assessment), a preliminary assessment (the cognitive function assessments and the questionnaire-based assessment), the lifestyle intervention, and a post hoc assessment (the cognitive function assessments and the questionnaire-based assessment). The cognitive function assessments refer to assessments to measure cognitive function, and the questionnaire-based assessment refers to an assessment to check lifestyle, personality, behavioral habit, and mental state (specifically, stress) using a questionnaire, for example. In the preliminary explanation, each subject was informed that the subject would be taking either the wasabi supplement or a placebo. The preliminary explanation, the preliminary assessment, and the post hoc assessment were each conducted in one day at the Institute of Development, Aging, and Cancer, Tohoku University. The lifestyle intervention was conducted for twelve weeks.

In the lifestyle intervention, the test formulation group took the test formulation and, the control formulation group took the control formulation. The subjects differed in whether to take the test formulation or the control formulation, but had the same duration of the lifestyle intervention and the frequency of intake (that is, daily). Although details of the assessments were common to all the subjects, the order of the assessments to be conducted differed from one subject to another in order to reduce the effect of the order of conducting the assessments and the effect of fatigue and the like due to undergoing the assessments.

### (2) Selection of Subjects

Seventy two healthy male and female elderly individuals (53 males: 19 females) who are right-handed native speakers of Japanese were selected as the subjects (mean age 65.43 years (SD=3.78)). It is known that dominant hand affects brain activity. It is common in brain imaging studies to include only right-handed subjects; thus only right-handed subjects were included in the same manner in the present Example 2. Those who did not meet the exclusion criteria described below were selected to be the subjects. For the exclusion criteria, the selection criteria of research (Nouchi et al., 2016: Nouchi et al., 2012), in which lifestyle intervention for elderly individuals was conducted, were referred to.

### Exclusion Criteria

Those with a history of a mental disorder, diabetes, a cranial nerve disease, or a heart disease (cardiac disease).
Those who are taking a drug that affects cognitive function (for examples, a benzodiazepine, an antidepressant, or a central nervous system agents).
Those who scored 26 points or less for MMSE (Mini Mental State Examination) which is a screening assessment for dementia.
Those with five points or more in an assessment to check the degree of depression (Geriatric Depression Scale).
Those who scored twelve points or less for Frontal assessment battery at bedside (FAB), which is a frontal lobe function assessment.
Those at risk of developing allergies due to ingestion of food (especially, those who are allergic to wasabi, milk, eggs, wheat, buckwheat noodles, peanuts, shrimps, or crabs).
Those with an alcohol consumption habit exceeding the average alcohol consumption of 60 g in the amount of pure alcohol (corresponding to three medium-size bottles of beer or three Gou of Japanese sake) per day on a weekly basis.
Those who are currently participating in another research apart from the present Embodiment 2, or those who have participated in another research within two months prior to the start of the test.
Those who are not able to write a lifestyle diary about whether the test formulation or the control preparation has been taken, and about the lifestyle intervention.

Prior to the start of the test, each subject was assigned a number, and a number list was created to randomly assign the subjects' numbers to either one of the two groups based on stratification by sex. Based on the list, the subjects were divided into one of the groups.

Based on an explanatory document approved by the Ethics Committee of Tohoku University, the purpose and safety of the test were explained in writing and verbally to the subjects, and then the subjects were admitted to participate in the test upon written consent of the subjects.

### (3) Lifestyle Intervention

In the test in Example 2, both groups took one capsule once a day for a total of twelve weeks. During the intake period, the subjects took one capsule of the test formulation or the control formulation with water or lukewarm water before bedtime. Whether the subject took the formulation was confirmed by means of a daily lifestyle diary. The daily lifestyle diary refers to a log to record of whether the formulation was taken and the time of intake, and to keep record of unusual events if any took place (for example, excessive exercise, traveling, or intake of medicine other than the test formulation or the control formulation). To which groups the subjects were assigned was concealed so that neither the subjects themselves nor the researchers would know until after the end of the test. During the lifestyle intervention, no special dietary restrictions were imposed, and the same lifestyles as before the intervention period were maintained.

The test formulation and the control formulation were provided to the researchers group by the applicant of the present application, KINJIRUSHI Co., Ltd. On packages of the formulations, characters were printed to identify the respective formulations. Then, employees at KINJIRUSHI Co., Ltd. who were not involved in the test prepared and sent each formulation. The researchers were informed only of how to identify the two types of formulations (identification by means of the characters printed on the bottom surface). Thus, the researchers were in a situation where the researchers were able to distinguish between the two formulations but did not know which one was the test formulation or the control formulation. The respective capsule-type formulations were in in the same type of color so as to be indistinguishable from the appearances, and provided from the researchers to the subjects. The test employed a double-blind method and was conducted such that which formulation groups the subjects were assigned to was concealed so that the researchers group conducting the test (namely, the principal researcher, co-researchers, and assignment personnel) and the subjects would not know until all the test and analyses were completed. After all the analyses were completed, the subjects' assignment information was unscaled.

### (4) Cognitive Function Assessment

In order to measure cognitive function, cognitive function assessments were conducted. The cognitive function assessments which will be described later were standardized cognitive function assessments to measure memory and other functions. All the cognitive function assessments were conducted by trained psychology examiners. The cognitive function assessments were conducted in the form of filling in answers using a pen and an assessment form to fill in the answers in the assessment. For the assessments using a pen and an assessment form, standardized assessments were used, namely “Logical Memory” and “Face-and-Name Associative Memory” for measuring episodic memory, and “Digit Span” for measuring working memory. Each assessment was conducted in a face-to-face manner. In each assessment, the time duration that the subjects took to answer was around five minutes.

### (5) Overview of Assessments

“Logical Memory”: It is an assessment to listen and memorize a story, and then recall the content of the story told. Both of an assessment to recall the story immediately after listening to the story (immediate) and an assessment to recall the story after a time interval of approximately 30 minutes (delay) were conducted.
“Face-and-Name Associative Memory”: It is an assessment to learn faces and names, and then answer the name corresponding to a face shown.
“Digit Span”: It is an assessment to, when a series of numbers is presented, answer the series of numbers in the reverse order (Backward Span).

### (6) Statistical Analysis

The amount of change in the scores of each assessment measured before and after the lifestyle intervention (post-intervention-pre-intervention) was calculated, and a statistical test was conducted using a permutation ANCOVA (analysis of covariance: analysis of covariance) regarding whether the lifestyle intervention produces a difference in effect. The scores before the intervention, age, sex, and the score in MMSE were used as covariates. All analyses were conducted using the statistical software R.

### (7) Results and Consideration

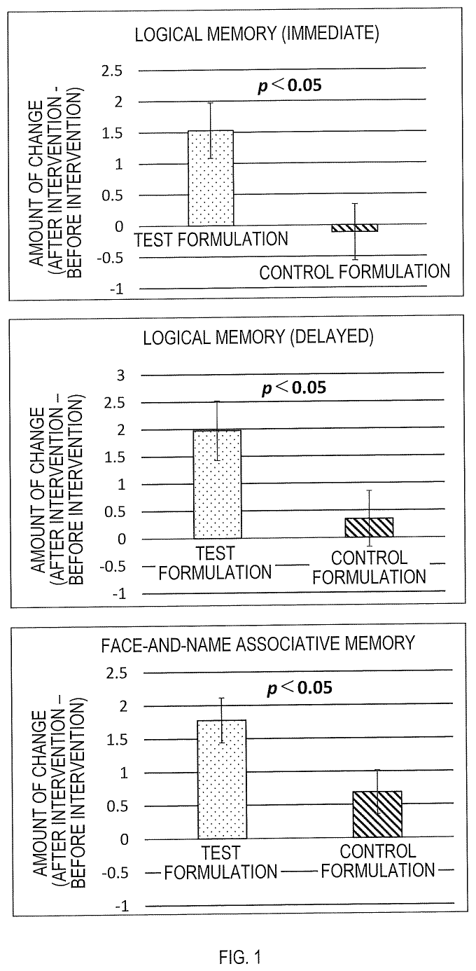

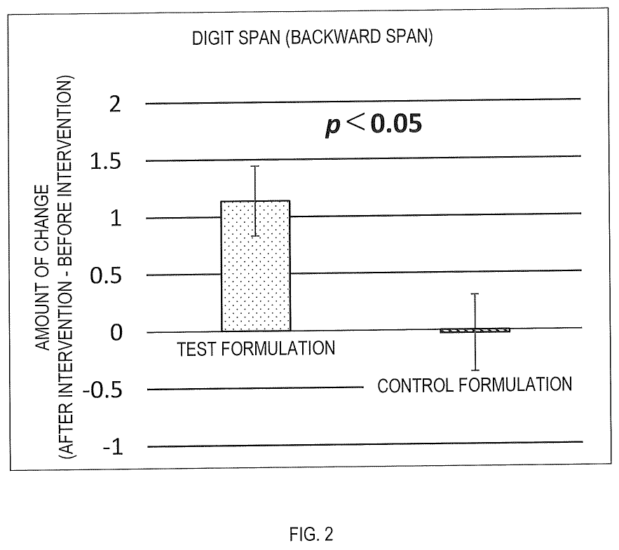

Score evaluation results of the Logical Memory (immediate), the Logical Memory (delay), and the Face-and-Name Associative Memory are shown in FIG. 1. Score evaluation results of the Digit Span (Backward Span) are shown in FIG. 2.

As a result of the analyses, in comparison between the test formulation group and the control formulation, the performance of the test formulation group was enhanced in the immediate recall and the delayed recall of the Logical Memory and the Face-and-Name Memory, which are indicators of episodic memory, and in the Backward Span, which is an indicator of working memory.

This suggests that episodic memory and working memory were enhanced due to administration of the test formulation. It has been reported that, in elderly individuals, when a task related to episodic memory is performed, DLPFC activity is involved in performance of the task (M. Natasha Rajah et.al., Brain., Volume 128, Issue 9, Pages 1964-1983 September 2005). Thus, it suggests that, in elderly individuals, intake of 6-MSITC increased blood flow in the DLPFC and thereby enhanced episodic memory.

### Example 3: Evaluation of Working Memory in Young Individuals

Using the test formulation and the control formulation prepared in Example 1, a single-dose administration test was conducted in the young individuals. Details are given below.

### (1) Flow of Test

In the test in Example 3, a preliminary assessment, a pre-intake assessment, administration, and a post-intake assessment (immediately after intake, 30 minutes after intake, and 60 minutes after intake) were conducted. On the day of a preliminary visit to the office, those whose informed consent were obtained and the eligibility to participate in the test were confirmed were registered as subjects for the test in Example 3. Then, the preliminary assessment was conducted. In the preliminary assessment, the cognitive function assessments were conducted. On a later examination day, the subjects took a formulation, and the pre-intake assessment and the post-intake assessment were conducted before and after the intake. There were two types of formulation for the subjects to take, namely the test formulation and the control formulation, out of which the subjects took one formulation once. In the pre-intake assessment and the post-intake assessment, a questionnaire-based assessment and measurement of brain activity were conducted using NIRS (Near-infrared spectroscopy).

### (2) Selection of Subjects

Forty healthy male and female individuals (age: 20 years old or over and 30 years old or under) who are right-handed native speakers of Japanese were selected as the subjects. For the same reason as in Example 2, only right-handed subjects were included in the present Example 3. Those who did not meet the exclusion criteria described below were selected to be the subjects. For the exclusion criteria, prior standards and others were referred to (Saito et al., 2018).

### Exclusion Criteria

Those with a history of a mental disorder, diabetes, a cranial nerve disease, or a heart disease (cardiac disease).
Those who are taking a drug that affects cognitive function (for examples, a benzodiazepine, an antidepressant, or a central nervous system agent).
Those who have difficulty in distinguishing red, blue, green, and yellow colors.
Those at risk of developing allergies due to ingestion of food (especially, those who are allergic to wasabi, milk, soybeans, eggs, wheat, buckwheat noodles, peanuts, shrimps, or crabs).
Those who are currently participating in another research apart from the present Example 3, those who have participated in another research within one month prior to the start of the research, or those who are scheduled to participate in another research during the research period.
Those who are determined to be inappropriate as a research subject by the principal researcher (medical doctor) or the co-researchers based on a comprehensive judgment on, for example, self-reported history of disease and supplement intake status.

Prior to the assessment day, each subject was assigned a number, and a number list was created to randomly assign the subjects' numbers to either one of the two groups based on stratification by sex. Based on the list, the subjects were divided into one of the groups.

Based on the explanatory document approved by the Ethics Committee of Tohoku University, the purpose and safety of the test was explained in writing and verbally to the subjects, and then the subjects were admitted to participate in the test upon written consent of the subjects.

### (3) Administration

The subjects took one capsule of the test formulation or the control formulation with water or lukewarm water.

The test formulation and the control formulation were provided to the researchers group by the applicant of the present application, KINJIRUSHI Co., Ltd. On packages of the formulations, characters were printed to identify the respective formulations. Then, employees at KINJIRUSHI Co., Ltd. who were not involved in the test prepared and sent each formulation. The researchers were informed only of how to identify the two types of formulations (identification by means of the characters printed on the bottom surface). Thus, the researchers were in a situation where the researchers were able to distinguish between the two formulations but did not know which one was the test formulation or the control formulation. The respective capsule-type formulations were in similar colors to be indistinguishable from the appearances, and provided from the researchers to the subjects. The test used a double-blind and was conducted such that which formulation groups the subjects were assigned to was concealed so that the researchers group conducting the test (namely, the principal researcher, co-researchers, and assignment personnel) and the subjects would not know until all the test and analyses were completed. After all the analyses were completed, the subjects' assignment information was unsealed.

### (4) Items and Method of Evaluation

In order to measure cognitive function, the cognitive function assessments were conducted. For the cognitive function assessments, “Raven's Matrices Test” and “Japanese Reading Ability Test”, which measure the overall cognitive function, were used. The Raven's Matrices Test is a test in which the subjects select, from among shape choices, a shape that matches the missing part in a diagram. The Japanese Reading Ability Test is a test in which the subjects answer the reading of Kanji characters. The overall cognitive function was measured only once per subject.

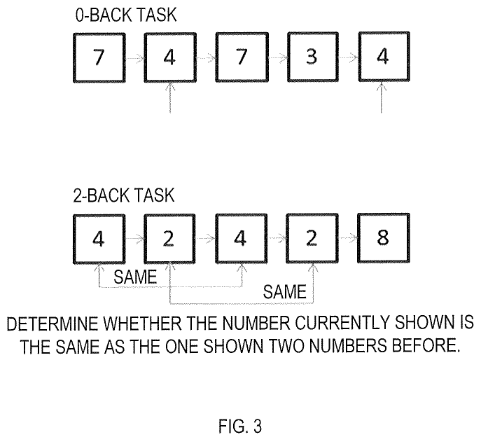

As a working memory task, an N-back task using characters was employed. In the N-back task, two tasks, 2-back and 0-back, were conducted (see FIG. 3).

In the 2-back task, it is determined, with regards to characters each presented at a two-second interval, whether the character currently presented is the same as the one presented two characters before. For example, in a case where the characters are presented in the order of 4, 2, 4, 2, and 8, since the third presented number “4” is the same as the first presented number “4”, a button is pressed at a point when the third number “4” is presented. Also, since the fourth presented number “2” is the same as the second presented number “2”, the button is pressed at a point when the fourth number “2” is presented. When number “8” is presented fifth, since the third presented number is “4” which is a different number, the button is not pressed.

In the 0-back task, with regards to characters presented at two-second intervals, it is determined whether a specified character is presented. For example, in a case where “4” is specified in advance, the subject presses the button when “4” is presented.

NIRS (HOT 1000, manufactured by NeU Corporation) was used to measure brain activity in the left dorsolateral prefrontal cortex (DLPFC) of the subjects while performing the N-back task. NIRS measures blood flow in the cerebral cortex using weak near-infrared light to measure brain activity. This enables measuring of brain activity in the DLPFC. During the measuring of brain activity, each task was conducted for 30 seconds, and there was a 30 second break between the tasks; in this cycle, alternation of the 2-back task and the 0-back task was repeated twice. This was each conducted before intake, 30 minutes after intake, and 60 minutes after intake of the formulation.

### (5) Statistical Analysis

Rate Correct Score (RCS) was calculated using the rate of correct answers and response time (that is, time until an answer) of each N-back task measured before intake, 30 minutes after intake, and 60 minutes after intake of the formulation. The RCS is an indicator which is integration of the rate of correct answers and the response time of the N-back tasks. In addition, a statistical test was conducted using a generalized linear mixed model (GLMM) regarding whether intake of the test formulation produces a difference in effect on the RCS and brain activity. The formulation (test formulation or control formulation) and assessment time (before intake, 30 minutes after intake, and 60 minutes after intake of the formulation) were used as fixed effects of the GLMM, the subjects were used as random effects, and age and sex were used as covariates.

### (6) Results and Consideration

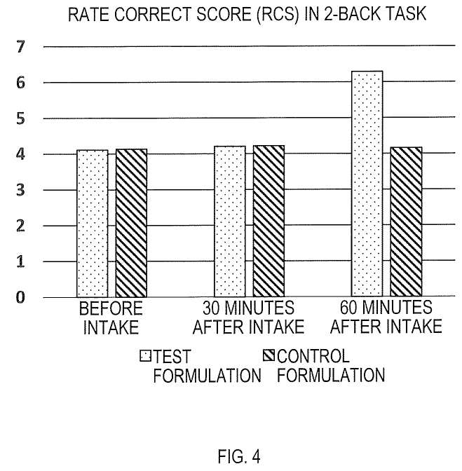

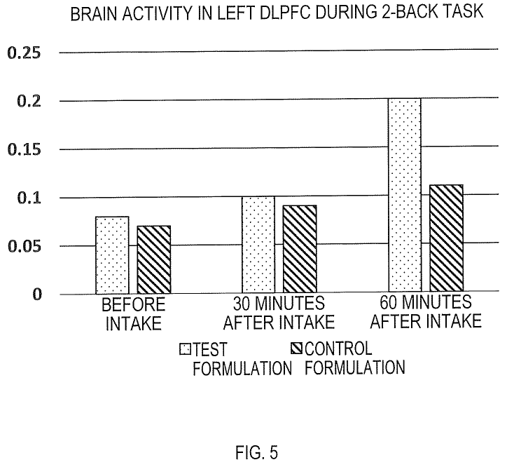

The calculation results of the RSC in the 2-back task are shown in FIG. 4, and the measurement results of brain activity in the left DLPFC are shown in FIG. 5.

In comparison between the test formulation group and the control formulation group in the young individuals, the performance of the test formulation group in the N-back task 60 minutes after intake of the formulation became better. Further, brain activity in the left DLPFC during the 2-back task was significantly enhanced in the test formulation group.

According to previous findings, it has been considered that cognitive function such as memory is enhanced because of an effect of inhibiting cellular damages caused by, for example, active oxygen (in other words, a long-term cell protection effect), and this effect is produced as an antioxidant effect is obtained by reducing production of active oxygen. In the present Example 3, by a single-dose administration of 6-MSITC contained in the wasabi extract to the young individuals, an immediate effect on working memory was first obtained 60 minutes after the administration. It is suggested that an increase in blood flow in the DLPFC is involved in the immediate effect.

1-12. (canceled)

13. A method for increasing blood flow in dorsolateral prefrontal cortex, the method comprising administering 6-methylsulfinylhexyl isothiocyanate to a subject.

14. The method according to claim 13, wherein the 6-methylsulfinylhexyl isothiocyanate improves and/or enhances cognitive function by increasing the blood flow in the dorsolateral prefrontal cortex.

15. The method according to claim 14, wherein the cognitive function is episodic memory.

16. The method according to claim 14, wherein the cognitive function is working memory.

17. The method according to claim 13, wherein the 6-methylsulfinylhexyl isothiocyanate is administered as a single dose.

18. The method according to claim 13, wherein the 6-methylsulfinylhexyl isothiocyanate is orally administered.

19. The method according to claim 13, wherein the 6-methylsulfinylhexyl isothiocyanate is extracted from wasabi or synthesized.

20. The method according to claim 13, wherein the 6-methylsulfinylhexyl isothiocyanate is incorporated into a food.

21. A method for increasing blood flow in dorsolateral prefrontal cortex, the method comprising administering a wasabi extract to a subject.

22. The method according to claim 21, wherein the wasabi extract is 6-methylsulfinylhexyl isothiocyanate and is incorporated into a food.

23. A method for activating dorsolateral prefrontal cortex, the method comprising administering 6-methylsulfinylhexyl isothiocyanate to a subject.

24. The method according to claim 23, wherein the 6-methylsulfinylhexyl isothiocyanate is incorporated into a food.
